(19) United States
(12) Patent Application Publication
(10) Pub. No.: US 2013/0072450 A1
(43) Pub. Date: Mar. 21, 2013

(54) ANTIOXIDATIVE AND HEPATOPROTECTIVE COMPOSITIONS CONTAINING DIARYLHEPTANOIDS FROM ALNUS JAPONICA

(71) Applicant: RNL BIO CO., LTD., Seoul (KR)

(72) Inventors: Jeong Chan RA, Gyeonggi-do (KR); Young Ho KIM, Daejeon (KR); Dong Hwan SOHN, Daejeon (KR)

(73) Assignee: RNL BIO CO., LTD., Seoul (KR)

(21) Appl. No.: 13/675,747

(22) Filed: Nov. 13, 2012

Related U.S. Application Data
(62) Division of application No. 12/774,761, filed on May 6, 2010.

(30) Foreign Application Priority Data
Dec. 14, 2009 (KR) 10-2009-0123863

(51) Int. Cl. A61K 31/12; A61P 1/16; A61K 31/7034

(52) U.S. Cl. 514/25; 514/679

(57) ABSTRACT

The present invention relates to antioxidative and hepatoprotective compositions, and more particularly to antioxidative and hepatoprotective compositions comprising diarylheptanoid compounds from Alnus japonica. The diarylheptanoid compounds from Alnus japonica include a compound selected from the group consisting of alusenone 1a, alusenone 1b, hirsutenone, hirsutanonol, oregonin, alnuside A, alnuside B, rubranoside B and rubranoside C. The antioxidative and hepatoprotective compositions have excellent antioxidative and hepatoprotective activities while having no side effects, and thus are useful for the prevention and treatment of oxidation-associated diseases and liver diseases.

### CROSS-REFERENCE TO RELATED APPLICATIONS

This application is filed according to the provisions of 35 U.S.C. §119(a) and claims the benefit of Korean Patent Application No. 2009-123863, filed on Dec. 14, 2009 in the Korean Intellectual Property Office, which is incorporated herein by reference in its entirety.

### BACKGROUND OF THE INVENTION

(a) Field of the Invention

The present invention relates to antioxidative and hepatoprotective compositions, and more particularly to antioxidative and hepatoprotective compositions containing diarylheptanoid compounds from Alnus japonica.

(b) Background of the Related Art

The liver is the chemical factory of the human body and plays important roles in producing and storing various proteins and nutrients required by the body and removing various substances harmful to the body.

The liver is located between the digestive system and the systemic circulatory system and plays an important role in protecting the human body from foreign toxic substances that entered the digestive system. For this reason, the liver is at a high risk of being exposed to toxic substances in addition to nutrients, and thus is more vulnerable to damage than other organs.

Liver diseases are classified according to their cause into viral liver diseases, alcoholic liver diseases, liver diseases by drug toxicity, fatty liver diseases, autoimmune liver diseases, metabolic liver diseases and other liver diseases. Liver diseases are difficult to diagnose in early stages owing to the absence of subjective symptoms, and thus are the leading causes of death in Korea and other countries. However, there is no effective therapeutic agent and diagnostic method for liver diseases.

In the Korea, people suffering from liver diseases are as many as liver diseases are the fifth leading cause of death among the whole population, and the first leading cause of death among people in their forties. Studies on agents for treating liver diseases have been actively conducted, but there is no drug showing the effect of inhibiting liver fibrosis in clinical trials. Thus, it is needed to develop economic and safe agents for treating liver diseases.

Many studies on the use of natural materials for the prevention and treatment of liver diseases have been conducted. Typical examples of such natural materials include silymarin isolated from the seeds of Silybum marianum, gomisin isolated from Schizandra chinensis, glycyrrhizin isolated from licorice root, and the like.

Free radicals have come to be appreciated for their importance to human health and disease. Many common and life-threatening diseases, including atherosclerosis, cancer and aging, have free radical reactions as an underlying mechanism of injury. One of the most common types of radicals is reactive oxygen species(ROS). These are the products of normal cell respiration and metabolism and are generally regulated by cellular defense systems present in the body. Such cellular defense systems reduce the amount of damage that free and reactive species radicals may cause by scavenging free radicals or enzymatically converting the free radicals to less toxic chemical species, thereby serving a physiological role as antioxidants.

Due to environmental agents such as pollution, and lifestyle factors such as smoking or exercising, the cellular exposure to free radicals is increased. Such increase may bring the body out of balance, especially as the body ages and the mechanisms that produce antioxidants or remove ROS are insufficient to counter oxidative stress. The body's antioxidant defense system can be impaired by the aging process and compromised, for example, by inflammation, arthritis, atherosclerosis, hypertension, radiation injury, ischemia, reperfusion injury, microbial infection, viral infection, burns/wound healing, tissue injury, sepsis, AIDS, diabetes, immunomodulative disorder, sickle cell anemia, the progression of cancer or neurological disorders, and many other disorders characterized by or caused in part by oxidative stress. The resulting damage can range from disruption of biological processes, killing of cells, and mutation of genetic material, which may lead to the occurrence of cancer. Accordingly, the potential use of antioxidant containing dietary supplements for protection against the effects of oxidative stress and the progressing of degenerative diseases and aging has been the subject of an increasing number of studies during the past four decades, see for example, Pauling., N Engl J. Med., Vt. C therapy of advanced cancer, Mar. 20, 1980; 302(12):694-5.

So, removal of free radical intermediates to retard or inhibit the oxidation process is required. Some powerful synthetic antioxidants were already developed (Shimizu, K. et al., Lipids, 36:1321, 2001) but were identified very powerful carcinogenic substances (Wichi, H. P. et al., Food Chem. Toxicol. 26:717-72, 1988). Because of this, health supplements from natural substances for use as antioxidants without side effects are strongly needed.

Meanwhile, Alnus japonica Steud. is a deciduous tree belonging to the Betulaceae family and has been used in folk remedies for treating fever, hemorrhage, diarrhea, alcoholism and the like (Lee S J, Korea folk medicine. Seoul National University Publishing Center Press, 40, 1996; Korean Patent Publication No. 2006023093).

Known compounds from Alnus japonica include diarylheptanoids, tannins, flavonoids, triterpenoids and the like (Kim H J et al., Arch Pharm Res, 28: 177179, 2005; Kuroyanagi M, et al., Chem Pharm Bull, 53: 15191523, 2005; Lee M W et al., Phytochemistry, 31: 28352839, 1992; Nomura M et al., Phytochemistry, 20: 10971104, 1981; Wada H et al., Chem Pharm Bull, 46: 10541055, 1998).

Among them, diarylheptanoids from different species of plants were reported to have anti-inflammatory, anticancer and antioxidative activities (Gonzalez-Laredo R F et al., J Nat Prod, 61: 1292-1294, 1998; Lee M W et al., Planta Med, 66: 551553, 2000; Lee W S et al., Planta Med, 71: 295299, 2005; Savikin K et al., Planta Med, 73: 980981, 2007; Joo S S et al., Planta Med, 74: 77, 2008).

Accordingly, the present inventors have made many efforts to isolate compounds having antioxidative and hepatoprotective activities from natural materials and, as a result, have found that diarylheptanoids including new alusenone from Alnus japonica have excellent antioxidative and hepatoprotective activities, thereby completing the present invention.

### SUMMARY OF THE INVENTION

It is an object of the present invention to provide an antioxidative composition from natural materials, which has excellent antioxidative effects while having no side effects.

Another object of the present invention is to provide a hepatoprotective composition from natural materials, which has excellent hepatoprotective effects while having no side effects.

To achieve the above objects, the present invention provides an antioxidative or hepatoprotective composition comprising at least one compound selected from the group consisting of diarylheptanoid compounds represented by the following formulas 1 to 4:

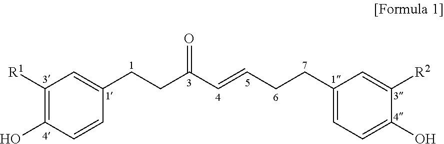

wherein R¹ is H and R² is OH, or R¹ is OH and R² is H;

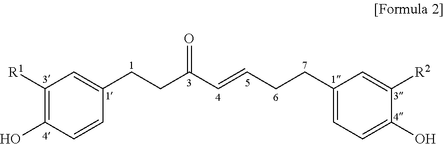

wherein R¹ and R² are OH;

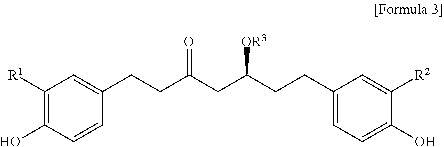

wherein R¹ and R² are each independently H or OH, and R³ is H, a β-D-xylopyranosyl group or a β-D-glucopyranosyl group; and

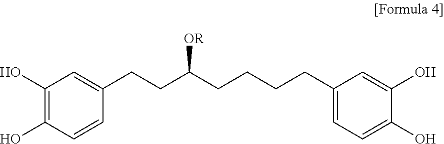

wherein R is a β-D-xylopyranosyl group or a β-D-xylopyranosyl-β-D-glucopyranosyl group.

In the present invention, the diarylheptanoid compounds are preferably derived from Alnus japonica.

Another object of the present invention is to provide methods of preventing and/or treating a disease related to an oxidative disorder or a liver disorder.

### BRIEF DESCRIPTION OF THE DRAWINGS

FIG. 1 is a schematic view showing the relationship between the H—H COSY and HMBC of an alusenone 1a compound according to an embodiment of the present invention.

### DETAILED DESCRIPTION OF THE PREFERRED EMBODIMENT

In one aspect, the present invention relates to an antioxidative composition comprising at least one compound selected from the group consisting of diarylheptanoid compounds represented by the following formulas 1 to 4:

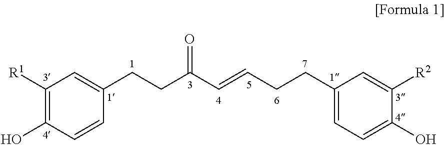

wherein R¹ is H and R² is OH, or R¹ is OH and R² is H;

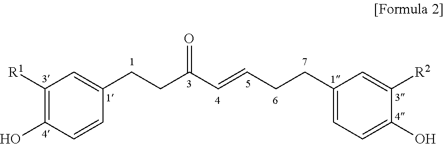

wherein R¹ and R² are OH;

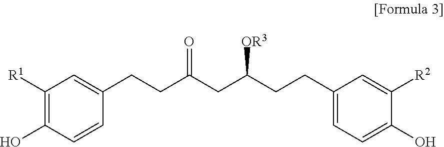

wherein R¹ and R² are each independently H or OH, and R³ is H, a β-D-xylopyranosyl group or a β-D-glucopyranosyl group; and

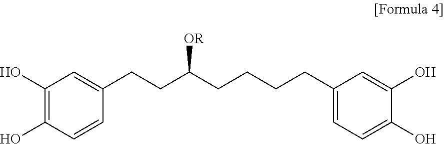

wherein R is a β-D-xylopyranosyl group or a β-D-xylopyranosyl-β-D-glucopyranosyl group.

In another aspect, the present invention relates to a hepatoprotective composition comprising at least one compound selected from the group consisting of diarylheptanoid compounds represented by the following formulas 1 to 4:

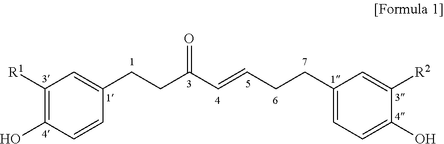

wherein R¹ is H and R² is OH, or R¹ is OH and R² is H;

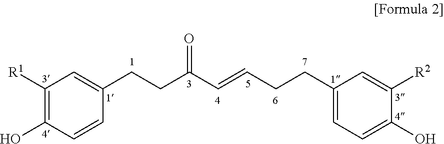

wherein R¹ and R² are OH;

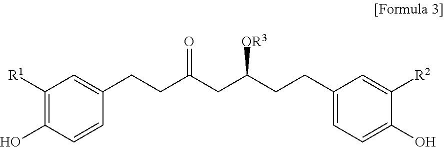

wherein R¹ and R² are each independently H or OH, and R³ is H, a β-D-xylopyranosyl group or a β-D-glucopyranosyl group; and

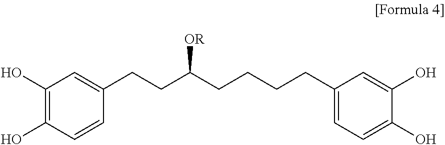

wherein R is a β-D-xylopyranosyl group or a β-D-xylopyranosyl-β-D-glucopyranosyl group.

In the present invention, the compounds of formula 1 can be exemplified by alusenone 1a and alusenone 1b represented by the following formulas, respectively:

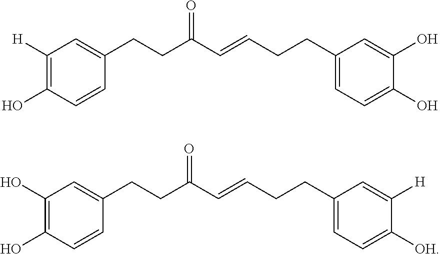 [SMILES: O=C(/C=C/CCc1ccc(O)c(O)c1)CCc1ccc(O)cc1 ; O=C(/C=C/CCc1ccc(O)cc1)CCc1ccc(O)c(O)c1]

In the present invention, the compounds of formula 2 can be exemplified by hirsutenone represented by the following formula:

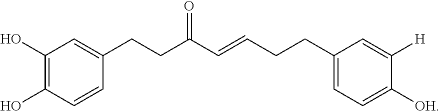 [SMILES: O=C(/C=C/CCc1ccc(O)cc1)CCc1ccc(O)c(O)c1]

In the present invention, the compounds of formula 3 can be exemplified by hirsutanonol, oregonin, alnuside A and alnuside B, represented by the following formulas, respectively:

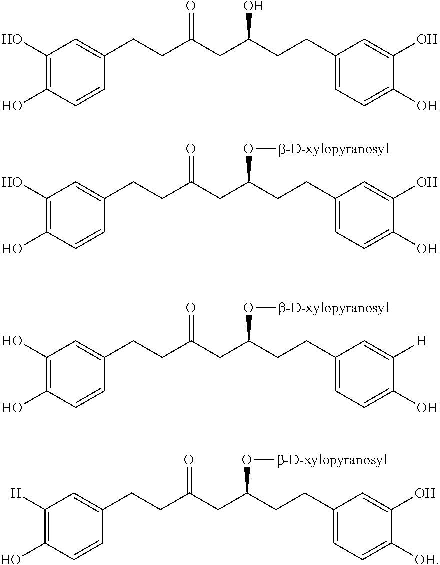

In the present invention, the compounds of formula 4 can be exemplified by rubranoside B and rubranoside C represented by the following formulas, respectively:

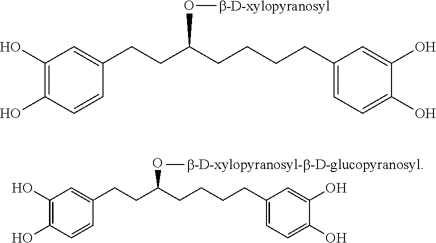

In the present invention, the diarylheptanoid compounds are preferably derived from Alnus japonica Steud.

The alusenone and other diarylheptanoid compounds can be obtained by a conventional method for isolating and purifying natural products.

A method of preparation of antioxidative composition comprising: (a) Distracting dried Alnus japonica samples by ethanol; (b) Eliminating dichloromethane fraction and n-butanol fraction; (c) Obtaining the fraction showing antioxidative activity; and (d) Preparing a composition containing the fraction according to step (c).

A method of preparation of hepatoprotective composition comprising: (a) Distracting dried Alnus japonica samples by ethanol; (b) Eliminating dichloromethane fraction and n-butanol fraction; (c) Obtaining the fraction showing hepatoprotective activity; and (d) Preparing a composition containing the fraction according to step (c).

Specifically, the dried bark of Alnus japonica is soaked in a solvent such as ethanol to obtain a crude extract of Alnus japonica. The crude extract of Alnus japonica is successively partitioned with organic solvents, such as dichloromethane, ethyl acetate and butanol, according to the polarity of the organic solvents.

Whether the solvent-partitioned layers have antioxidative or hepatoprotective activity is examined, and then the ethyl acetate layer having the highest activity is subjected to silica gel column chromatography and reverse-phase silica gel column chromatography to separate the active ingredients.

The antioxidative or hepatoprotective composition according to the present invention can be used in a method for preventing and/or treating a disease related to an antioxidative disorder or a liver disorder by administering the composition. The antioxidative disorder may be selected from the group consisting of: inflammation; infection; atherosclerosis; hypertension; cancer; radiation injury; neurological disease; neurodegenerative disease; ischemia/reperfusion injury; aging; wound healing; glutathione deficiency; acquired immunodeficiency syndrome; sickle cell anemia; and diabetes mellitus. The liver disorder may be selected from the group consisting: viral liver diseases; alcoholic liver diseases; liver diseases by drug toxicity; fatty liver diseases; autoimmune liver diseases; and metabolic liver diseases. Liver disease may be selected from the group consisting of hepatitis, cirrhosis, fatty liver and liver cancer.

The antioxidative or hepatoprotective composition according to the present invention may comprise the diarylheptanoid compound from Alnus japonica as an active ingredient alone or in combination with other components. Also, the composition of the present invention may additionally comprise one or more pharmaceutically acceptable carriers, excipients and/or diluents according to the formulation, mode of administration, and intended use thereof. If the active ingredient is provided as a mixture, it may be contained in the antioxidative or hepatoprotective composition in an amount of 0.001-99.9 wt %, but is preferably contained in an amount of 0.1-50 wt %.

The antioxidative or hepatoprotective composition containing the diarylheptanoid compound from Alnus japonica as an active ingredient may be formulated for oral administration (for example, oral dietary supplement) in the form of powders, granules, tablets, capsules, suspensions, emulsions, syrups or aerosols according to a conventional method. It may be formulated in the form of external preparations, suppositories or sterile injectable solutions. Carriers, excipients or diluents that can be included in the composition containing the compound include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrolidone, water, methyl hydroxybenzoate, propyl hydroxybenzoate, talc, magnesium stearate and mineral oils. For formulations, commonly used diluents or excipients such as fillers, expanders, bonding agents, wetting agents, disintegrants and surfactants, etc., are used. Solid formulations for oral administration include tablets, pills, powders, granules, capsules, etc. Such solid dosages are prepared by admixing the compound of the present invention with at least one excipient, such as starch, calcium carbonate, sucrose or lactose, gelatin, etc. In addition to simple expedients, lubricants such as magnesium styrate, talc, etc. may be added. Liquid formulations for oral administration, such as suspensions, internal solutions, emulsions, syrups, etc., may comprise simple diluents, e.g., water and liquid paraffin, as well as various excipients, e.g., humectants, sweeteners, aromatics, preservatives, etc. Formulations for parenteral administration include sterilized aqueous solutions, non-aqueous solvents, suspensions, emulsions, lyophilized agents, suppositories, etc. Non-aqueous solvents and suspensions may be prepared using vegetable oils, such as propylene glycol and polyethylene glycol, olive oil, or using injectable esters such as ethyloleate. As a base for suppositories, Witepsol, Macrogol, Tween 61, cacao fat, laurin fat, glycerogelatin, etc. may be used.

The preferred dosage of the antioxidative or heptaprotective composition containing the diarylheptanoid compound from Alnus japonica may vary depending on the patient's conditions and weight, the severity of illness, the type of formulation, administration route and the duration of treatment, but may be selected appropriately by a person skilled in the art. However, for desired effects, the compound of the present invention may be administered in a daily dosage of 0.0001 to 100 mg/kg, and preferably 0.001 to 10 mg/kg. The daily dosage may be taken in a single dose, or may be divided into several doses. However, the dosage does not in any way limit the scope of the present invention.

### EXAMPLES

Hereinafter, the present invention will be described in further detail with reference to examples. It is to be understood, however, that these examples are for illustrative purposes only and are not to be construed to limit the scope of the present invention.

### Example 1

### Extraction of Alnus Japonica Sample

The bark of A. japonica was collected in Yanzi Province, China, in September, 2006, and was taxonomically identified. A voucher specimen (CNU 08102) has been deposited at the College of Pharmacy, Chungnam National University, Korea. The dried sample (3.0 kg) was crushed, soaked in 9 L of 95% ethanol at room temperature and extracted three times, and the extract was filtered and concentrated under reduced pressure to evaporate the solvent. The resulting ethanol extract (900 g) was suspended in 4 L of water, and successively partitioned with 4 L of dichloromethane (CH₂Cl₂), ethyl acetate (EtOAc) and n-butanol (BuOH), thus obtaining a dichloromethane fraction (139 g), an ethyl acetate fraction (400 g) and an n-butanol fraction (35 g).

### Experimental Example 1

### Experiment on Antioxidative Activities of A. Japonica Sample Fractions

In order to measure the antioxidative activities of the A. japonica sample fractions, the radical scavenging capacities of the fractions were measured in the following manner using the total oxyradical scavenging capacity (TOSC) assay. The TOSC assay was performed by a modification of the method proposed by Winston et al. (Free Radic. Biol. Med., 24: 480˜493, 1998) (J. Agric. Food Chem. 56, 10510-10514, 2008).

Peroxyl radicals were generated by the thermal homolysis of 2,2′-azobisamidinopropane (ABAP) at 35° C., and peroxynitrite was generated by the spontaneous decomposition of SIN-1.

It was reported that the generated reactive oxygen species react with α-keto-γ-methiolbutyric acid (KMBA) to produce ethylene gas and that this reaction does not change with temperature within a certain range.

Then, a total volume of 1 ml of the reaction solution was placed in a 10-ml vial sealed with a rubber septum. For detection of the produced ethylene gas, a gas chromatograph (GC), equipped with a DB-05 capillary column and a flame ionization detector) (FID), was used, an oven set was 60° C., and the injector and the detector were set at 180° C. As a mobile phase, helium was injected into the column at a rate of 2 ml/min. For quantitative determination of the amount of ethylene gas in the vial, 150 μl of gas was sampled from the vial with a gas-tight syringe using a headspace technique and injected into the injector.

The area under the kinetic curve (AUC) was obtained by integrating the graph determined from the results of the above experiment, and the TOSC value was calculated according to the following equation 1 and shown in Table 1 below:

TOSC=100−(∫SA/∫CA×100) [Equation 1]

wherein ∫SA=integrated area from the curve of the sample reaction, and

∫CA=integrated area from the curve of the control reaction.

TABLE 1

| | | TOSC value (% inhibition) | |
|---|---|---|---|
| Extract | C (mg/mL) | Peroxyl radical | Peroxynitrite |
| EtOH | 0.01 | 85.6 ± 3.2 | 65.8 ± 1.4 |
| | 0.05 | 89.0 ± 2.4 | 74.1 ± 1.6 |
| CH₂Cl₂ | 0.01 | 33.2 ± 1.8 | 23.4 ± 1.1 |
| | 0.05 | 66.5 ± 2.3 | 47.5 ± 1.7 |
| EtOAc | 0.01 | 87.7 ± 1.2 | 76.6 ± 1.8 |
| | 0.05 | 88.9 ± 1.8 | 85.6 ± 0.9 |
| n-BuOH | 0.01 | 86.7 ± 1.6 | 56.3 ± 1.0 |
| | 0.05 | 85.3 ± 2.0 | 72.9 ± 1.1 |

As can be seen in Table 1 above, the ethanol and ethyl acetate (EtOAc) fractions had excellent antioxidative activities.

### Experimental Example 2

### Experiment on Hepatoprotective Activities of A. japonica Sample Fractions

In order to measure the hepatoprotective activities of the A. japonica sample fractions, the primarily cultured hepatocytes of white rats were isolated, and then the inhibitory abilities of the fractions against necrosis induced by treating the isolated hepatocytes with 1.2 mM of tert-butyl hydroperoxide for 50 minutes were examined (Park E J et al., Planta Med, 71: 508513, 2005).

Hepatocytes were isolated from white rats according to the method of Seglen et al. Briefly, white rats were anesthetized and the abdomen was incised. Then, a catheter was inserted into the portal vein and perfused with Ca²⁺, Mg²⁺-free Hank's balanced salt solution bubbled with a mixed gas of 5% CO₂ and 95% O₂ at 37° C. so as to remove blood from the liver. Then, the catheter was perfused with 0.05% type IV collagenase and 1 mM CaCl₂ solution. The liver tissue was removed from the body, and a hepatocyte suspension was prepared from the liver tissue and centrifuged at 50×g for 2 minutes. The precipitated hepatocytes were collected and washed twice with Ca²⁺, Mg²⁺-free Hank's balanced salt solution. The washed hepatocytes were added at a concentration of 1×10⁶ cells/ml to Williams' Medium E (WME, GibcoBRL, USA) containing 10% (v/v) fetal bovine serum (FBS, GibcoBRL), 10⁻⁸ M insulin, 50 U/ml of penicillin and 50 μg/ml of streptomycin (Sigma) in an incubator coated with type I collagen, and were then cultured under 5% CO₂ at 37° C. After 2 hours of the culture, the cells were washed with Hank's balanced salt solution to remove non-adhered cells, and the medium was replaced with fresh medium. Then, the cells were cultured under 5% CO₂ at 37° C. for 16 hours and then used in the experiment.

### Example 2

### Isolation and Identification of Diarylheptanoid Compounds from A. japonica

100 g of the ethyl acetate fraction determined to have excellent antioxidative and hepatoprotective activities in Experimental Examples 1 and 2 was subjected to silica gel column chromatography (12×10 cm, 63˜200 μm) with a chloroform-methanol gradient solvent system (15:1 v/v to 0:1 v/v; 1 L for each solvent) to afford six fractions (1a˜f). Fraction 1a (1.5 g) was subjected to reverse-phase YMC silica gel column chromatography (2×35 cm, 40˜63 μm) with water and methanol (1:1; 1.5 L) to afford a mixture of alusenone 1a and alusenone 1b (1:1; 12.5 mg) and hirsutenone (550 mg, yellow syrup). Fraction 1b (1.3 g) was subjected to reverse-phase silica gel column chromatography (2×35 cm, 40˜63 μm) with water and methanol (6:5; 1.2 L), and then to silica gel column chromatography (1.5×30 cm, 40˜63 μm) with a chloroform-methanol gradient solvent system (10:1, 1 L), thus obtaining hirsutanonol (15.3 mg, colorless syrup). Fraction 1d (5.1 g) was subjected to reverse-phase silica gel column chromatography (5×30 cm, 40˜63 μm) with water and methanol (6:5, 2 L) to afford alnuside A (13.6 mg, colorless syrup), alnuside B (18.1 mg, colorless syrup) and four sub-fractions (2a˜d). Sub-fraction 2a (1.4 g) was subjected to silica gel column chromatography (21.5×30 cm, 40˜63 μm) with chloroform-methanol-water (50:10:1; 1.3 L) to afford oregonin (630 mg, colorless syrup). Sub-fraction 2b (0.58 g) was subjected to silica gel column chromatography (1.5×30 cm, 40˜63 μm) with dichloromethane-methanol-water (70:10:1; 1.1 L) to afford platyphyllone (25.7 mg, colorless syrup) and platyphylloside (10.3 mg, colorless syrup). Sub-fraction 2c (0.32 g) was subjected to silica gel column chromatography (1×30 cm, 40˜63 μm) with ethylacetate-methanol-water (200:10:1; 1 L) to afford platyphyllonol-5-xylose (18.4 mg, colorless syrup). Finally, sub-fraction 2d (0.85 g) was subjected to reverse-phase silica gel column chromatography (2×30 cm, 40˜63 μm) with water and methanol (6:5; 1.5 L) and to silica gel column chromatography (1×30 cm, 40˜63 μm) with chloroform-methanol-water (75:10:1; 1.2 L), thus obtaining rubranoside B (5.3 mg, colorless syrup) and rubranoside C (8.2 mg, colorless syrup).

The isolated compounds were analyzed by mass spectrometry and NMR, and the analysis results showed that the active component was alusenone of formula 1. The spectral data of the compound are as follows.

Alusenone (1a, 1b): UV (MeOH)λₘₐₓ (log ε) 204.0 (2.04), 250.0 (2.49); IR (film) νₘₐₓ 3396, 1644, 1435, 1042 cm⁻¹; ¹H NMR (CD₃OD, 500 MHz) and ¹³C NMR (CD₃OD, 125 MHz)(see Table 2); FABMS (positive ion mode) m/z 313 [M+H]⁺; HREIMS (positive ion mode) m/z 312.1355 [M]⁺ (calculated for C₁₉H₂₀O₄ 312.1361).

TABLE 2

| | 1a | | 1b | |
|---|---|---|---|---|
| | δ_{C} | δ_{H} | δ_{C} | δ_{H} |
| Position | (125 MHz, CD₃OD) | (500 MHz, CD₃OD) | (125 MHz, CD₃OD) | (500 MHz, CD₃OD) |
| 1 | 30.84 | 2.77 (2H, overlapped) | 31.09 | 2.72 (2H, t, J = 7.0) |
| 2 | 42.90 | 2.79 (2H, t, J = 7.0) | 42.85 | 2.79 (2H, t, J = 7.0) |
| 3 | 203.00 | | 203.05 | |
| 4 | 131.72 | 6.06 (1H, dt, J = 16.0, 1.0) | 131.81 | 6.06 (1H, dt, J = 16.0, 1.0) |
| 5 | 149.46 | 6.85 (1H, m) | 149.39 | 6.89 (1H, m) |
| 6 | 35.79 | 2.46 (2H, m) | 35.90 | 2.46 (2H, m) |
| 7 | 34.94 | 2.62 (2H, t, J = 7.5) | 34.72 | 2.66 (2H, t, J = 7.5) |
| 1′ | 133.34 | | 134.15 | |
| 2′ | 130.52 | 6.99 (1H, d, J = 7.5) | 116.66 | 6.62 (1H, d, J = 1.5) |
| 3′ | 116.32 | 6.69 (1H, d, J = 7.5) | 144.65 | |
| 4′ | 156.77 | | 146.32 | |
| 5′ | 116.32 | 6.69 (1H, d, J = 7.5) | 116.47 | 6.67 (1H, d, J = 8.0) |
| 6′ | 130.52 | 6.99 (1H, d, J = 7.5) | 120.83 | 6.50 (1H, dd, J = 8.0, 1.5) |
| 1″ | 133.96 | | 133.18 | |
| 2″ | 116.66 | 6.61 (1H, d, J = 1.5) | 130.45 | 6.98 (1H, d, J = 7.5) |
| 3″ | 144.68 | | 116.31 | 6.69 (1H, d, J = 7.5) |
| 4″ | 146.36 | | 156.81 | |
| 5″ | 116.47 | 6.65 (1H, d, J = 8.0) | 116.31 | 6.69 (1H, d, J = 7.5) |
| 6″ | 120.71 | 6.48 (1H, dd, J = 8.0, 1.5) | 130.45 | 6.98 (1H, d, J = 7.5) |

^{a}The data were assigned on the basis of H-H COSY, HMQC, and HMBC experiments. The chemical shifts δ are in ppm, coupling constant J in Hz in parentheses.
[Formula 1]

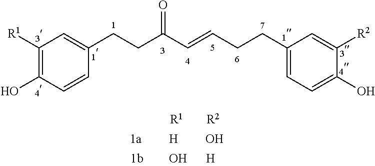

Alusenone 1a and alusenone 1b of formula 1 were obtained in the form of a mixture (1:1 ratio). Alusenone 1a and alusenone 1b were analyzed by FABMS (m/z 313, [M+H]⁺) and HREIMS (found at m/z [M]⁺ 312.1355, calculated for C₁₉H₂₀O₄ 312.1361) and, as a result, the molecular weights of alusenone 1a and alusenone 1b were all the same as the molecular weight of C₁₉H₂₀O₄.

Although most of the NMR signals appeared double, indicating a close structural similarity between alusenone 1a and alusenone 1b, COSY, HMBC and HMQC experiments were allowed to pick the signals for alusenone 1a and alusenone 1b individually.

The ¹H NMR and ¹³C NMR spectra of alusenone 1a and alusenone 1b exhibited seven carbon chains linked to each other in two different aromatic ring systems, and these carbon chains are the typical structures of diarylheptanoids (Table 2).

One aromatic ring is substituted with hydroxy at the 3,4 positions like the case of hirsutanonol, and the other aromatic ring is symmetrically substituted with hydroxy at the para-position like the case of platyphyllone.

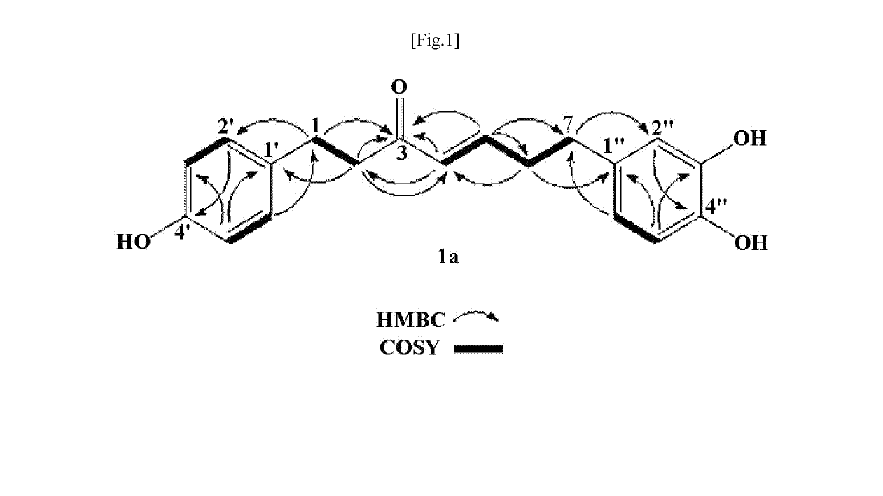

By carbon signals at δ 203.00 (C-3), 149.46 (C-5) and 131.72 (C-4) in alusenone 1a, and δ 203.05 (C-3), 149.39 (C-5), and 131.81 (C-4) in alusenone 1b, it is suggested that the heptane chains form α,β-unsaturated ketone. Also, the C-4 carbon signals in alusenone 1a and alusenone 1b were determined by HMQC related to the same proton signal δ 6.06 (dt, J=16.0, 1.0 Hz) indicating the E form of C-4 and C-5 double bonds. Furthermore, the partial structures thereof were determined by the COSY and HMBC spectra (FIG. 1).

Because the positions of the two phenyl groups (4-hydroxy phenyl and 3,4-dihydroxy phenyl in alusenone 1a and alusenone 1b are similar to those of alnuside A and aluside B, it is suggested that alusenone 1a and alusenone 1b are two isomers. It was reported that alusenone 1b [(4E)-1-(3,4-dihydroxyphenyl)-7-(4-hydroxyphenyl)-heptene-3-one] was isolated from Alnus rubra Bong (Chen J et al., Planta Med, 64: 74˜75, 1998) and Amomum muricarpum Elmer (Giang P M et al., Chem Pharm Bull, 54: 139˜140, 2006). However, alusenone 1a [(4E)-1-(4-dihydroxyphenyl)-7-(3,4-hydroxyphenyl)-heptene-3-one] has not yet been isolated.

10 other diarylheptanoid compounds (see Table 3) were confirmed to be hirsutenone, hirsutanonol, oregonin (Lee M W et al., Arch Pharm Res, 23: 5053, 2000), alnuside A, alnuside B, platyphyllonol-5-xylose, platyphyllone, platyphylloside (Smite E et al., Phytochemistry, 32: 365˜369, 1993), rubranoside B and rubranoside C (Gonzalez-Laredo R F et al., Nat Prod Lett, 13: 75˜80, 1999), based on known physico-chemical properties, NMR spectra (¹H and ¹³C-NMR), and ESI-MS. Among these compounds, hirsutenone and oregonin were obtained as major compounds.

### Experimental Example 3

### Experiment on Antioxidative Activities of Diarylheptanoid Compounds from A. japonica

The antioxidative activities of the diarylheptanoid compounds from A. japonica were measured by the TOSC (Total Oxyradical Scavenging Capacity) assay in the same manner as in Experimental Example 1, and the measurement results are shown in Table 3.

TABLE 3

| | Peroxyl radical | | | | Peroxynitrite | | | |
|---|---|---|---|---|---|---|---|---|
| | TOSC of | | | Curve fit | TOSC of | | | Curve fit |
| Comp. | 20% | 50% | 80% | r² | 20% | 50% | 80% | r² |
| alusenone | 0.38 | 2.95 | 22.98 | 0.9822 | 0.50 | 7.29 | 105.33 | 0.9963 |
| 1a, 1b | | | | | | | | |
| mixture | | | | | | | | |
| hirsutenone | 0.31 | 1.58 | 8.19 | 0.9916 | 0.18 | 2.11 | 24.16 | 0.9973 |
| hirsutanonol | 0.36 | 1.86 | 9.64 | 0.9971 | 0.42 | 6.12 | 87.32 | 0.9891 |
| oregonin | 0.44 | 1.79 | 7.30 | 0.9828 | 0.57 | 5.76 | 58.58 | 0.9855 |
| alnuside A | 1.15 | 5.34 | 24.87 | 0.9949 | 1.07 | 17.99 | >200 | 0.9981 |
| alnuside B | 1.18 | 5.45 | 25.95 | 0.9849 | 1.05 | 18.29 | >200 | 0.9981 |
| Platyphyllone-5- | 6.28 | 40.52 | >200 | 0.9927 | 3.96 | 45.05 | >300 | 0.9954 |
| xylose | | | | | | | | |
| platyphyllone | 4.30 | 24.25 | >200 | 0.9861 | 3.15 | 35.42 | >300 | 0.9828 |
| platyphylloside | 4.81 | 38.94 | 136.69 | 0.9896 | 3.79 | 45.79 | >300 | 0.9853 |
| rubranoside B | 0.65 | 2.48 | 9.48 | 0.9920 | 0.52 | 5.27 | 53.53 | 0.9887 |
| rubranoside C | 0.55 | 2.38 | 10.27 | 0.9909 | 1.16 | 6.17 | 32.75 | 0.9935 |
| Trolox | 1.42 | 5.58 | 21.92 | 0.9879 | 3.49 | 9.73 | 27.14 | 0.9914 |
| Curcumin | 0.62 | 3.36 | 18.39 | 0.9896 | 1.06 | 7.05 | 46.96 | 0.9986 |
| Quercetin | 0.22 | 1.64 | 12.38 | 0.9841 | 2.45 | 8.67 | 30.65 | 0.9967 |

As can be seen in Table 3 above, hirsutenone, hirsutanonol and oregonin had the highest scavenging capacity for peroxyl radicals and peroxynitrite, and rubranoside B, rubranoside C, the alusenone 1a/1b mixture, alnuside A and alnuside B had the second highest scavenging capacity. In comparison with positive controls for peroxyl radicals, hirsutenone, hirsutanonol and oregonin had antioxidative activities higher than those of trolox and curcumin, and their antioxidative activities were approximately similar to those of flavonol having potent antioxidative activity and the antioxidant quercetin. The alunosene 1a/1b mixture showed a scavenging activity for peroxyl radicals, which was higher than that of trolox, but lower than those of curcumin and quercetin.

Generally, with respect to the scavenging capacities for peroxyl radicals and peroxynitrite, most of the compounds had relatively low inhibition ratios (TOSC of 20%). Interestingly, hirsutenone had higher inhibition rates (TOSC of 50% and 80%) for two kinds of oxyradicals than that of trolox.

Interestingly, all the other compounds require higher concentrations for peroxynitrite than for peroxyl radicals. Such results suggest that these compounds have half-lives and reactivities which are different between two kinds of oxyradicals, and it is believed that, because peroxyl radicals have higher stability and lower reactivity than peroxynitrite, they can be more easily scavenged (Regoli F et al., Toxicol Appl Pharmacol, 156: 96˜105, 1999; Halliwell B et al., Food Chem Toxicol, 33: 601˜617, 1995).

Hirsutenone, hirsutanonol, oregonin, rubranoside B and rubranoside C, which comprise two 3,4-dihydroxyphenyl rings in their structures, have higher scavenging activities against reactive oxygen species (ROS) than the alusenone 1a/1b mixture, alnuside A and alnuside B, which comprise 3,4-dihydroxyphenyl and 4-hydroxyphenyl rings.

Platyphyllone-5-xylose, platyphyllone and platyphylloside, which comprise two 4-hydroxyphenyl rings, are considered to have weak activities against reactive oxygen species.

### Experimental Example 4

### Experiment on Hepatoprotective Activities of Diarylheptanoid Compounds from A. japonica

In the same manner as in Experimental Example 2, the hepatoprotective activities of the diarylheptanoid compounds from A. japonica were measured by measuring their inhibitory activities against necrosis induced by treating the primarily cultured hepatocytes of rats with 1.2 mM of tert-butylhydroperoxide (tBH) for 50 minutes, and the measurement results are shown in Table 4 below. Herein, tBH is metabolized into free radical intermediates, which initiate lipid peroxidation to affect the cell integrity, and release LDH into the medium, thus forming covalent bonds with cellular molecules causing damage to the cells.

TABLE 4

| Compound | ED₅₀(μM)^{a} |
|---|---|
| alusenone 1a, 1b mixture | 8.44 ± 0.23 |
| hirsutenone | 3.08 ± 0.14 |
| hirsutanonol | 6.28 ± 0.81 |
| oregonin | 19.19 ± 0.67 |
| alnuside A | 26.49 ± 0.73 |
| alnuside B | 25.92 ± 0.33 |
| platyphyllone-5-xylose | ND^{b} |
| platyphyllone | ND^{b} |
| platyphylloside | ND^{b} |
| rubranoside B | 9.81 ± 0.18 |
| rubranoside C | 12.83 ± 0.46 |
| Silibinin^{c} | 130.90 ± 6.70 |

^{a}Results are the means ± SE of three independent experiment in triplicate
^{b}Not determined
^{c}Positive control

As can be seen in Table 4, the alusenone 1a/1b mixture, hirsutenone, hirsutanonol, oregonin, alnuside A, alnuside B, rubranoside B and rubranoside C showed LDH release inhibitory rates (ED₅₀) of 8.44, 3.08, 6.28, 19.19, 26.49, 25.92, 9.81 and 12.83 μM, respectively, but platyphyllone-5-xylose, platyphyllone and platyphylloside had no effect on the inhibition of LDH release.

Silibinin (purity >98%, Sigma Chemical Co., St Louis, USA) known to have excellent hepatoprotective activity was used as a control and found to have an LDH release inhibitory rate (ED₅₀) of 130.90 μM (Modriansk M et al., Gen Physiol Biophys, 19, 223˜235, 2000).

The above results suggest that the diarylheptanoid compounds from A. japonica have hepatoprotective activity in addition to antioxidative activity.

### Experimental Example 5

### Experiment on Toxicity of Diarylheptanoid Compounds from A. japonica

In order to examine the acute toxicities of the diarylheptanoid compounds from A. japonica according to the present invention, the following experiment was carried out.

12 male and 12 female 4-week-old specific pathogen-free ICR mice (3 male mice and 3 female mice per dose group were bred in an animal facility at a temperature of 22±3° C. and a humidity of 55±10% under a 12-hr light/12-hr dark cycle. The mice were acclimated for about one week before use in the experiment. The experimental animals were given food (CJ CheilJedang Corp., Korea, for mice and rats) and sterile water ad libitum.

Each of the diarylheptanoid compounds from A. japonica, prepared in the above Example, was dissolved in 0.5% Tween 80 as a solvent at a concentration of 50 mg/ml, and then administered orally to each mouse at doses of 0.04 ml (100 mg/kg), 0.2 ml (500 mg/kg) and 0.4 ml (1,000 mg/kg) per 20 g of weight of the mouse. Each sample was administered orally once, and for 7 days after the administration of each sample, the mice were observed for side effects or fatality. Specifically, on the day of administration, changes in general symptoms and the presence or absence of dead animals were observed 1 hr, 4 hr, 8 hr and 12 hr after administration, and for a period of one day to seven days after administration, changes in general symptoms and the presence or absence of dead animals were observed at least once in the morning and afternoon each day. Also, at 7 days after administration, the animals were sacrificed and dissected, and the internal organs were examined. The change in weight of the animals was measured every day from the day of administration in order to determine whether the weight of the animals was reduced due to the diarylheptanoid compounds from A. japonica.

As a result, in all the mice administered with the test compounds, there were no specific clinical symptoms and also no dead mice. Also, in weight changes, blood examination, blood biochemical examination, biopsy finding, etc., no change in toxicity was observed. Accordingly, the diarylheptanoid compounds from A. japonica according to the present invention showed no change in toxicity up to a dose of 1,000 mg/kg in all the mice, and were confirmed to be safe substances having an oral lethal dose (LD₅₀) of at least 1,000 mg/kg.

### Experimental Example 6

### Preparation of Antioxidative or Hepatoprotective Compositions Containing Diarylheptanoid Compounds from A. japonica

A. Preparation of Powder

Alusenone (1a: 150 mg, 1b: 150 mg): 300 mg

Lactose: 100 mg

Talc: 10 mg

Powder was prepared by mixing the above components and filling the mixture into a sealed packaging material.

B. Preparation of Tablet

Alusenone (1a: 25 mg, 1b: 25 mg): 50 mg

Corn starch: 100 mg

Lactose: 100 mg

Magnesium stearate: 2 mg

A tablet was prepared by mixing the above components and tableting the mixture.

C. Preparation of Capsule

Alusenone (1a: 25 mg, 1b: 25 mg): 50 mg

Corn starch: 100 mg

Lactose: 100 mg

Magnesium stearate: 2 mg

According to a conventional method, a capsule was prepared by the above components and filling the mixture into a gelatin capsule.

The inventive compositions containing alusenone or other diarylheptanoid compounds from A. japonica have excellent antioxidative and hepatoprotective activities while having no side effects, and thus are useful for the prevention and treatment of oxidation-associated diseases and liver diseases.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1.-9. (canceled)

10. A method for preventing and/or treating a disease related to liver disorder, which comprises administering a hepatoprotective composition comprising at least one compound selected from the group consisting of diarylheptanoid compound represented by the following formulas 1 to 4: 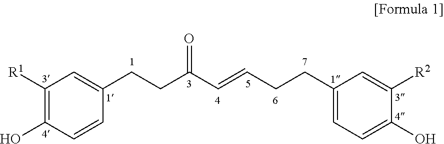
wherein R¹ is H and R² is OH, or R¹ is OH and R² is H; 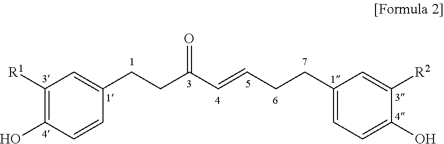
wherein R¹ and R² are OH; 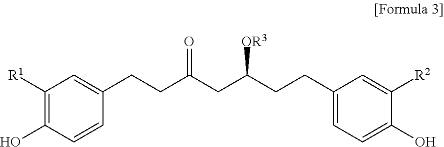
wherein R¹ and R² are each independently H or OH, and R³ is H, a β-D-xylopyranosyl group or a β-D-glucopyranosyl group; and 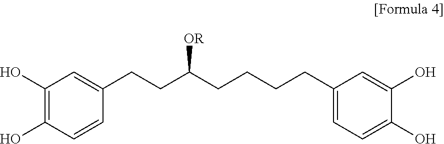
wherein R is a β-D-xylopyranosyl group or β-D-xylopyranosyl-β-D-glucopyranosyl group.

11. The method according to claim 10, wherein the liver disorder is selected from the group consisting of: viral liver diseases; alcoholic liver diseases; liver diseases by drug toxicity; fatty liver diseases; autoimmune liver diseases; and metabolic liver diseases.

12. The method according to claim 10, wherein the composition is prepared as an oral dietary supplement.

13. The method according to claim 10, wherein the diarylheptanoid compound is derived from Alnus japonica.

14. The method according to claim 10, wherein the diarylheptanoid compound is selected from the group consisting of 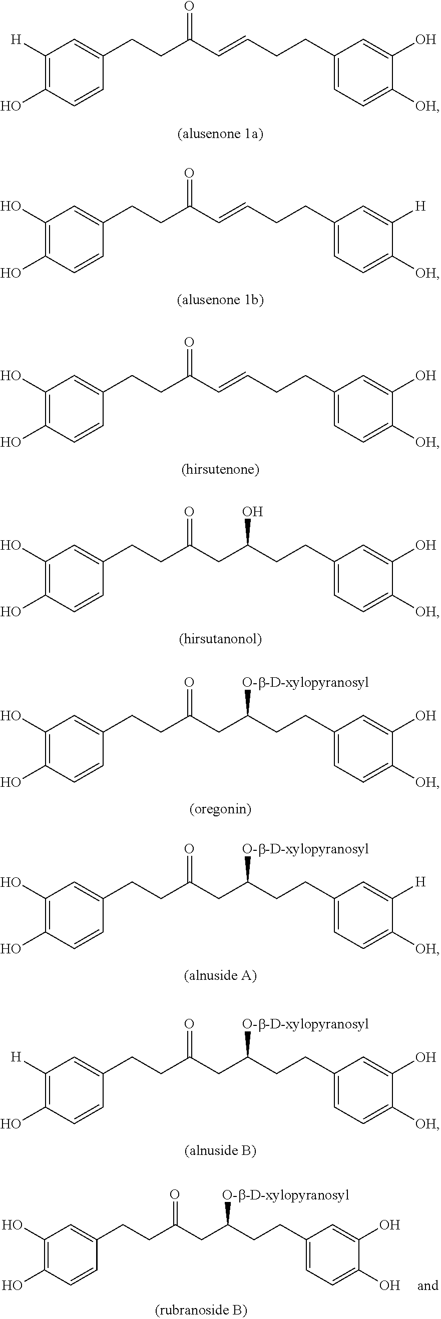 [chemical structure]
